(19) United States
(12) Patent Application Publication
(10) Pub. No.: US 2014/0303699 A1
(43) Pub. Date: Oct. 9, 2014

(54) Unibody Head Gear Personal Temperature Moderating Device

(71) Applicant: Lawrence Harley Wahl, Franklin, TN (US)

(72) Inventor: Lawrence Harley Wahl, Franklin, TN (US)

(21) Appl. No.: 13/859,326

(22) Filed: Apr. 9, 2013

(51) Int. Cl. A61F 7/08; A42B 1/02; A42B 3/12; A42B 1/00

(52) U.S. Cl. CPC A61F 7/08; A42B 1/008; A42B 1/02; A42B 3/12; A61F 2007/0008
USPC 607/110

(57) ABSTRACT

The unibody head gear personal temperature moderating device has the form of a baseball cap or beanie or helmet which is itself constructed from a thermal retention pack or packs. The thermal retention pack or packs constitute the inner and outer walls of the baseball cap or beanie or helmet while the cooling or warming gel resides between the inner and outer walls. The inner wall of the thermal retention packs is the surface which comes in contact with the wearer's head. The outer wall of the thermal retention pack or packs constitutes the exterior of the head apparel. The entire baseball cap or beanie or helmet may be placed in a freezer or warmer to thermally charge it for use.

### BACKGROUND OF THE INVENTION

The present invention relates generally to a personal temperature moderating device, and more particularly to a personal temperature moderating device capable of being worn on the head, and even more particularly to a personal temperature moderating device shaped like a baseball cap or beanie or helmet which is capable of being cooled or warmed in a freezer or warmer then placed on the wearer's head, and even more particularly to a personal temperature moderating device which is constructed in a unibody design from a thermal retention pack that comprises both the inner and outer walls of the device while storing the cooling gel in between the two walls.

Wearing head apparel, whether required or for aesthetic purposes, often results in an increase in the wearer's body temperature, or at the very least challenges the body's natural cooling mechanisms. This can lead to overheating and the adverse health consequences intertwined with such a condition. For exemplary purposes, consider the effects of wearing one specific type of head apparel, a cotton or wool baseball cap. A full coverage baseball cap inhibits the natural convection of heat away from the wearer's head. During winter months, choosing to wear a baseball cap for aesthetic or work requirements prevents the wearing of stocking caps or other warming caps. Consequently, the effects of a full coverage cap, in conjunction with the potentially extreme ambient temperatures and extreme level of physical exertion not uncommon in athletic and workplace endeavors, can readily lead to discomfort, premature fatigue, poor decision making, heat illness, and injury.

Incorporating an effective personal temperature moderating device in head apparel is not a simple task. Prior art has disclosed several elaborate technical schemes based upon various forms of cooling tubes, fans, air conditioners, and other complex approaches which would be very difficult and expensive for the designer to manufacture and for the wearer to afford and manage. The challenge is even greater in head apparel such as a baseball cap or beanie or helmet which is expected to be very lightweight and simple to manage. In such a scenario, the personal temperature moderating device will have to work around the constraints dictated by the relatively small size of a baseball cap or beanie or helmet and the expectation that the baseball cap or beanie or helmet will not be excessively heavy or encumber the movement of the wearer with attached cables, fans, hanging straps or flanges and, if applicable, any rules promulgated by regulatory bodies that certify the safety of baseball cap or beanie or helmet for use in sports and workplace situations, as any personal temperature moderating device that deteriorates the function, comfort, or manageability of the baseball cap or beanie or helmet will not be acceptable.

However, regardless of the specific type of head apparel contemplated, what is needed is a personal temperature moderating device that can be easily donned and removed and comfortably worn to help moderate the wearer's body temperature—whether it is desired to cool or warm the wearer. This is not a predicament that the prior art has left unaddressed. A myriad of previous attempts have been proffered to solve the dilemma.

U.S. Pat. No. 5,327,285 issued on Jul. 12, 1994, to Edward J. Karlan discloses an elongated, semi-flat, tubular body constructed of a flexible fluid impervious material. The tubular body may hold a eutectic solution that is suitable for absorbing heat when the tubular body is placed proximate the wearer's head. The patent further discloses positioning the tubular body in a pouch or a sleeve along the crown of a hat. Accordingly, this positions the tubular body so that the eutectic solution may conduct the heat away from the wearer's head to keep the wearer cool.

U.S. Pat. No. 6,233,745 issued on May 22, 2001 to Lorin Dean Friesen discloses a flexible neck cover that is attached to the lower portion of a cap or hat and is formed to drape over the wearer's neck and shoulders. Additionally, the neck cover may be saturated with water or include a water dispensing vial to provide an evaporative cooling effect for the wearer.

U.S. Pat. No. 6,185,750 issued Feb. 13, 2001 to Dexter Reginald Dumas discloses a cooling device that is adapted to fasten around a wearer's neck and also attach to the lower portion of a cap, if desired. The cooling device has a pouch that can be filled with ice so as to cool the wearer.

U.S. Pat. No. 6,857,126 issued on Feb. 22, 2005 to Jed Cowell discloses a personal cooling device adapted for suspension from the rear strap of a cap. The device includes an elongated pouch member configured to hold a coolant pack or, alternatively, a pouch member with separated compartments designed to hold individual coolant packs so that the coolant does not pool in the lower region of the pouch. The personal cooling device is then suspended from the rear of the cap to allow the pouch, and coolant contained therein, to be proximate the neck and rear head of the wearer.

The above described prior art discloses various attempts to cool a person wearing head apparel. However, each is deficient when compared with the present invention. This is especially the case when the head apparel is a baseball cap or beanie or helmet designed to provide cooling or warming to the wearer. The prior art does not disclose an apparatus that can be easily or quickly removed, re-charged, and replaced on the head in a fashion that allows real-world implementation, where time and manageability are of the essence in arenas such as the workplace and sports. Furthermore, the prior art does not disclose an apparatus which could effectively achieve the stated goal of providing easily attainable, meaningful cooling to a wearer. Thus, what is needed is a personal temperature moderating device, preferably a baseball cap or beanie or helmet, which effectively cools or warms the wearer, is reusable, and is a practical easy to manage, non-cumbersome implementation. The prior art is devoid of an apparatus capable of satisfying the aforementioned aims.

### BRIEF SUMMARY OF THE INVENTION

The present invention is directed at a personal temperature moderating device that is capable of attenuating changes in the wearer's body temperature. In one embodiment of the present invention, the personal temperature moderating device is a baseball cap which is itself a gel filled pack such that the baseball cap itself is constructed in a unibody design from gel pack elements allowing the baseball cap to remain lightweight and easily handled while still effectively cooling or warming the wearer. In this embodiment, the fluid impervious exterior and interior walls of the baseball cap are the fluid impervious exterior and interior walls of the gel pack itself, with temperature moderating gel in between the two walls. The complete sealed assembly of the exterior and interior walls with the temperature moderating gel inside, also known as a thermal retention or gel pack, comprises the unibody head gear personal temperature moderating device.

The personal temperature moderating device is a baseball cap or beanie or helmet which itself is constructed in the form of a thermal retention pack. It is a unibody design in that the inner and outer walls of the device provide the device's structure while containing the temperature moderating gel within. The entire baseball cap or beanie or helmet is charged to a desired temperature (either above or below the ambient temperature) by placing it in a refrigerator or warmer. The inner layer or inner wall of the baseball cap or beanie or helmet is positioned against the wearer's head and provides a comfortable engagement between the device and the wearer. This inner layer of the baseball cap or beanie or helmet also constitutes the inner layer of the thermal retention pack. The outer layer of the device, which also constitutes the top of the baseball cap or beanie or helmet, constitutes the outer layer of the thermal retention pack. This upper or outer portion is a flexible or hard plastic which may be coated on the outside with cotton or other materials to create the aesthetically pleasing appearance generally associated with baseball cap or beanie or helmets and in certain embodiments to provide a hard outer shell for impact protection.

When the wearer dons the baseball cap or beanie or helmet, which itself a thermal retention pack, the thermal energy flows out of the pack to the wearer. This process either cools or warms the wearer depending on the initial thermal state of the retention pack.

Importantly, the personal temperature moderating device is designed so that it can be readily accommodated by the shape of the wearer's head—in one preferred embodiment this includes a baseball cap. Preferably, the personal temperature moderating device will be easily placed on or removed from the head to allow recharging of the thermal retention packs once they have been depleted. Such recharging may encompass placing the device in cooling or warming equipment such as a refrigerator or warmer for a predetermined amount of time and then placing the device back onto the head. Accordingly, the present invention discloses a personal temperature moderating device designed to either cool or warm the wearer as demanded by existing environmental conditions and circumstances.

It is an additional object of the present invention to provide a personal temperature moderating device that is amenable to being very easily managed without encumbrance of tubes or cables hanging down from the head to be connected to other portions of a cooling apparatus and is capable of making the wearer more comfortable while maintaining an aesthetically pleasing appearance.

It is an additional objective of the present invention to provide a personal temperature moderating device that is easily useable in the real world since it is very easily and inexpensively cooled or warmed in a typical refrigerator or microwave oven.

It is yet another objective of the present invention to provide a personal temperature moderating device capable of being expeditiously donned on the wearer's head in about one second and very easily removed from the wearer's head in about one second and replaced into the cooling or warming apparatus without encumbrance of tubes or cables or separate internal cooling elements which would necessitate a complex process of removal or placement of the personal temperature moderating device.

Finally, it is an object of the present invention to provide a personal temperature moderating device that is capable of being recharged and reused once the moderating device's cooling or heating capabilities have been depleted.

### BRIEF DESCRIPTION OF THE SEVERAL VIEWS OF THE DRAWINGS

FIG. 1 is an overhead view of the personal temperature moderating device.

FIG. 2 is a bottom view of the personal temperature moderating device.

FIG. 3 is a bottom view illustrating the gel pack panel or panels that comprise the crown of the baseball cap.

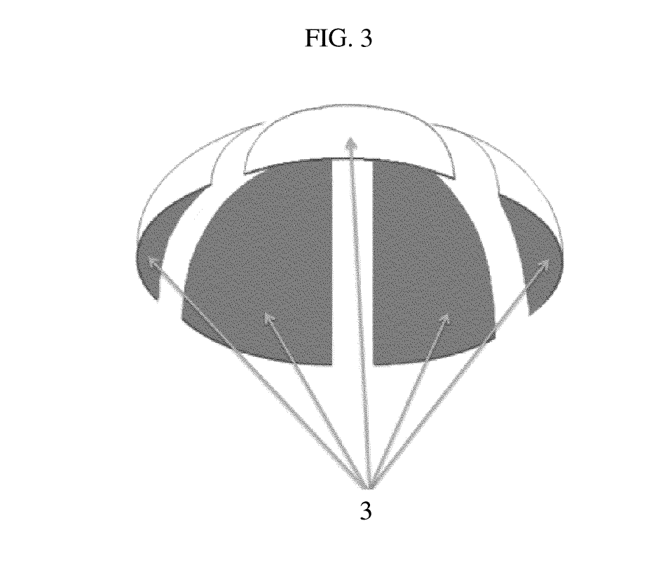

FIG. 4 is an expanded view of one of the gel pack panels showing the composition of that panel.

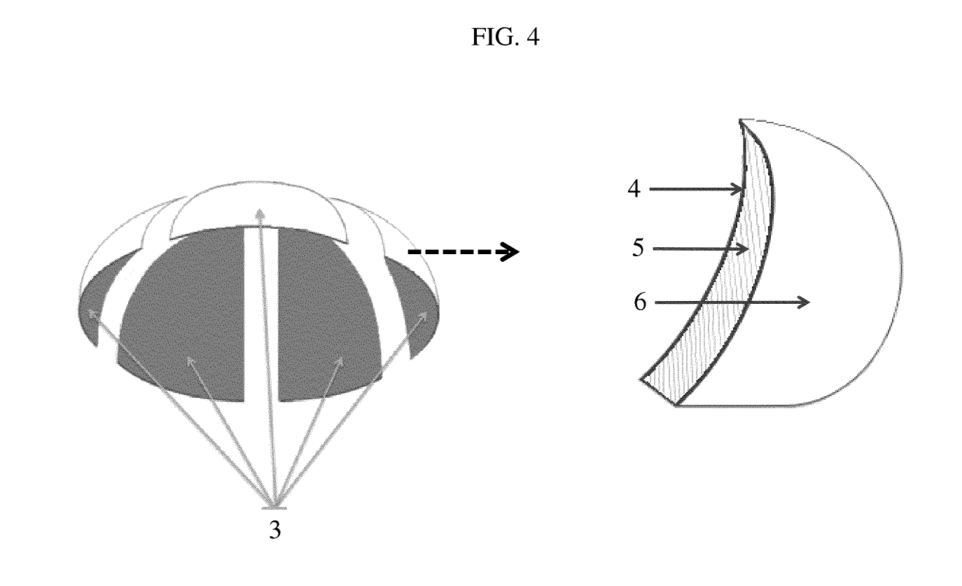

FIG. 5 shows one preferred embodiment of the personal temperature moderating device in the form of a beanie with or without external attachments such as spinners, ears, or fans.

### DETAILED DESCRIPTION OF THE INVENTION

The present invention relates generally to personal temperature moderating devices for use as head apparel. More specifically, the present invention relates to a personal temperature moderating device in the form of a baseball cap or beanie or helmet where the device itself is constructed in a unibody fashion from a thermal gel pack or packs. The device is configured so that it can be easily and quickly received onto the head and then worn to provide thermal benefit to the wearer until the thermal benefit ends and the device is removed and exchanged for a thermally charged device of the same design. The device, which is proximate to the wearer's head, is able to distribute the thermal energy, emanating from the thermal retention pack, to the wearer's head so that the device can help to either cool or warm the wearer, depending on the initial thermal state of the thermal packs.

In one preferred embodiment directed at helmets having anti-trauma pads, such as football helmets, hockey helmets, cycling helmets, and lacrosse helmets, the thermal retention pack is fabricated so that it constitutes the helmet's hardened exterior and interior anti-trauma pad. In this embodiment, the outer layer of the thermal retention pack is a hardened plastic to provide impact protection while the inner layer of the thermal retention pack is in direct contact with the wearer's head. In this case, impact attenuation is performed by the thermal retention pack while it provides the desired cooling or warming.

In another preferred embodiment directed at beanie caps, the beanie cap is itself is constructed from the thermal retention pack. In this embodiment, the outer layer of the thermal retention pack may be coated with a fabric to create a pleasing aesthetic, while the inner layer of the thermal retention pack aligns and comes in direct contact with the wearer's head.

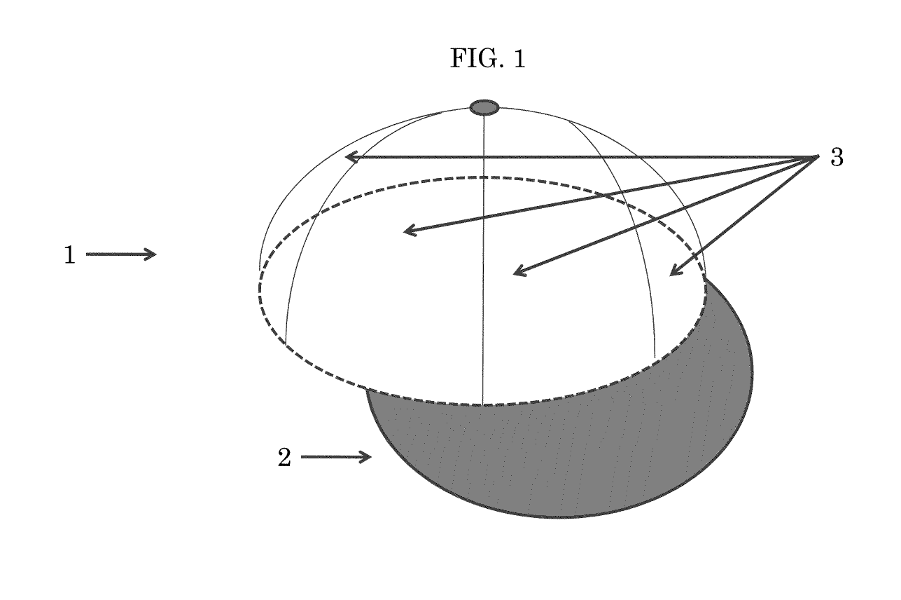

Now referring to the figures. FIG. 1 is a top view of the personal temperature moderating device, in this embodiment a baseball cap, showing the crown 1 or upper portion of the device comprising a single or multiple thermal retention packs 3. These thermal retention pack 3 contains an aqueous solution 5, a solid medium or some amalgamation of the two. Preferably the thermal retention pack 3 will be sufficiently pliable so that it may conform to the shape of the wearer's head. The brim 2 of the baseball cap is shown as well to illustrate perspective.

The thermal retention pack 3 is heat welded together or formed together or molded as one piece, to form the crown 1 of the personal temperature moderating device. More specifically, the thermal retention pack 3 is created when the outer layer 6 is welded at the seams to the inner layer 4, then filled with thermal retention gel 5. Thus, the crown 1 of the baseball cap is itself one large thermal retention pack, otherwise known as a gel pack.

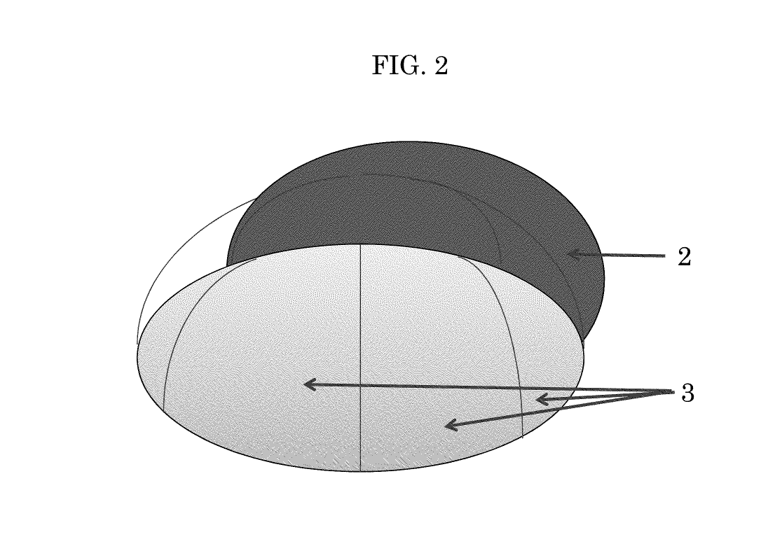

FIG. 2 is a bottom view of the baseball cap. The inner concave surface 3 of crown 1 comes directly in contact with the wearer's head. It is this contact which facilitates the transfer of thermal energy via thermal conductivity. In the case of head cooling, the cold inner surface 3 draws heat to itself while conducting cooling energy to the wearer's cranium.

Preferably, the inner surface 3 will be made of a urethane or polyurethane material, or have an insulative coating, such as, but not limited to, cotton, poly weave, and/or a synthetic fabric. However, it would be obvious to one of ordinary skill in the art that a plethora of other materials comport with the strictures placed on the inner surface 3.

The middle layer 5 is a thermal retention liquid or gel. The Invention also envisions using liquid and solid coolants and other substances that may be used to retain heat and warm the wearer.

As shown in FIG. 1, the personal temperature moderating device is designed so that a wearer may readily and comfortably don the head apparel without concern about proper alignment and fit on the head, as might be the case if separate gel pack inserts were used.

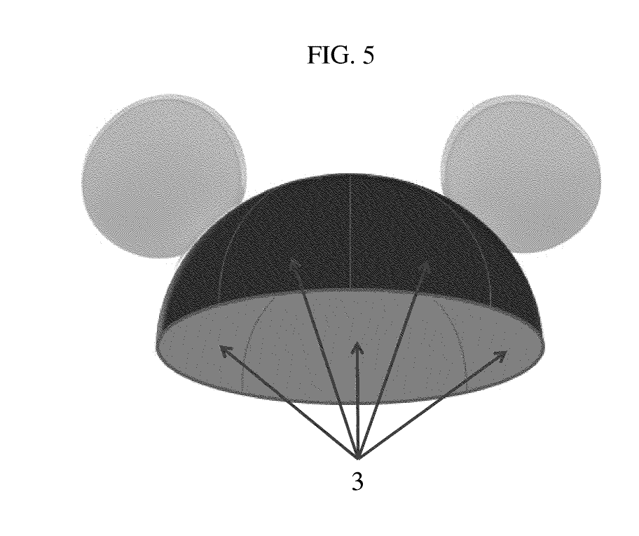

As shown in FIG. 5, a preferred embodiment of the personal temperature moderating device is in the form of a beanie which is a unibody construction of the thermal retention pack 3, wherein the outer layer of the thermal retention pack 3 is urethane or polyurethane covered in black cotton for aesthetic purposes, and the inner layer is urethane or polyurethane which comes in direct contact with the wearer's head.

As is now manifest from the proceeding disclosure, the features of the personal temperature moderating device allow it to be received onto the wearer's head and effectively moderate the temperature of the wearer. Moreover, in the preferred embodiment, the personal temperature moderating device is received onto the wearer's head while allowing the wearer to enjoy the various forms of protection from radiated heat or impact trauma to the head provided by the personal temperature moderating device.

Advantageously, the above-described configuration promotes the ends of the invention; namely, it provides for a personal temperature moderating device. If a wearer dons the temperature moderating device, the wearer's head would engage the inner surface 3. Furthermore, assuming the wearer desires to be cooled, the temperature moderating gel, pre-cooled to a temperature below that of the ambient conditions, would facilely conduct its thermal energy via the inner surface 3 to the wearer's cranium.

Thus it is seen that the personal temperature moderating device of the present invention readily achieves the ends and advantages mentioned as well as those inherited therein, although certain preferred embodiments of the invention have been illustrated and described for purposes of the present disclosure. Numerous changes may be made by those skilled in the art which changes are encompassed within the spirit and scope of the present invention and defined by the appended claims.

1. A personal temperature moderating device, for use as head apparel, comprising:
an outer layer which serves as the physical exterior of the head apparel, wherein the outer layer provides a hard or soft external surface of the head apparel;
an inner layer which serves as the inside of the head apparel in direct contact with the top of the wearer's head, wherein the inner layer has a thermal conductivity; and
a middle layer disposed between the outer layer and the inner layer, wherein the middle layer comprises a thermal retention liquid or gel capable of being cooled or warmed.

2. The personal temperature moderating device of claim 1 wherein the outer layer or exterior of the head apparel may be manufactured from hard or soft plastic to provide impact protection to the head apparel wearer.

3. The personal temperature moderating device of claim 1 wherein the middle gel layer of the device may deform under a compressive force to attenuate the force of an impact to the head apparel and head.

4. The personal temperature moderating device of claim 1 wherein the outer layer or exterior of the head apparel may be coated with fabric or other textiles to create an aesthetically pleasing appearance.

5. The personal temperature moderating device of claim 1 wherein the outer layer and inner layer are heat welded or otherwise assembled into a unibody assembly at their seams to allow them to be filled with thermal retention liquid or gel.

6. The personal temperature moderating device of claim 1 wherein the exterior layer of the device is not a separate part from the thermal retention pack.

7. The personal temperature moderating device of claim 1 wherein the complete assembly comprising the outer layer, inner layer, and middle layer of thermal retention liquid or gel, and any required brims or visors or accessories comprise the head apparel itself.
